(19) United States
(12) Patent Application Publication
(10) Pub. No.: US 2008/0058767 A1
(43) Pub. Date: Mar. 6, 2008

(54) TUBAL CANNULATOR AND METHODS OF USE

(76) Inventors: Carlos A. Rotman, Lake Bluff, IL (US); Nasir Rana, Oakbrook, IL (US); Winfried Reich, Nashville, TN (US); Edgardo Yordan, Deerfield, IL (US)

Correspondence Address: WILLIAM DOUGLAS HARE, 3 ANDERSON LANE, PRINCETON, NJ, 08540, US

(21) Appl. No.: 11/851,227

(22) Filed: Sep. 6, 2007

Related U.S. Application Data
(60) Provisional application No. 60/824,759, filed on Sep. 6, 2006.

(51) Int. Cl. A61M 25/00

(52) U.S. Cl. 604/523

(57) ABSTRACT

The invention relates to a cannulator that has an adjustable shape for conforming to an anatomy to reach the tubal ostium of a patient. The cannulator includes a body segment, a tip section, and a handle. The body segment is made of a malleable material having a malleable characteristic with a degree of resistance to lateral deflection until sufficient force is applied to cause permanent bending thereby imparting a shape to the body segment. The tip section is positioned at a distal end of the body segment and the handle is positioned at a proximal end of the body segment.

### CROSS-REFERENCE TO RELATED APPLICATIONS

This application is a nonprovisional application claiming priority from U.S. Provisional Patent Application No. 60/824,759, filed on Sep. 6, 2006, the contents of which are incorporated herein by reference in their entirety.

### TECHNICAL FIELD

The field of the invention generally relates to an improved tubal cannulator for cannulating the fallopian tubes and facilitating tubal reconnection after, for example, tubal ligation.

### BACKGROUND

Referring to FIGS. 1 and 2, the female reproductive system includes the fallopian tubes 100, the uterus 105, the uterine corneal 107, the ovary 110, the cervix 115 and the vagina 120. The fallopian tube 100 is a narrow muscular organ connecting the uterus 105 to the ovary 110. The inner tubal lining of the fallopian tube 100 is rich in cilia, microscopic hair-like projections that beat in waves and move the egg to the uterus. The fallopian tube is about 10 cm (4 inches) in length and is made up of several segments. The interstitial segment 125 arises from the uterus 105 at the tubal ostium 127 and passes through the uterine muscle to the isthmic segment 130. The isthmic segment 130 is a narrow muscular segment by the uterus 105 that is between the interstitial segment 125 and the ampullary segment 135. The ampullary segment 135 is a wider middle segment that is between the isthmic segment 130 and the infundibular segment 140. The infundibular segment 140 is a funnel shaped segment near the ovary. Extending from the infundibular segment 140, the fimbrial segment 145 has a ciliary lining that faces the ovary.

There are a number of different medical conditions that can result in damage to the fallopian tubes. These conditions include tubal blockage and tubal scarring. Physicians treat medical conditions of the fallopian tubes using a number of procedures. For example, in patients with fallopian tubes occluded near the origin (i.e., a proximal occlusion) and resulting infertility, physicians cannulate the fallopian tubes. Physicians also facilitate tubal reconnection due to complete obstruction of the tubal lumen after tubal ligation or pathologic process. These interventions occur when the patients seek reversal of sterilization and/or treatment of “tubal factor infertility.”

Tubal obstructions are divided into those that are a result of mucous plugs and those with true obstructions that are secondary to scar tissue and have total obliteration of the tubal lumen. The first type of tubal obstruction can be overcome by pushing or flushing the tubal plug, or by advancing a stent and dislodging the plug. The second type of tubal obstruction typically necessitates surgery to resect the occluded portion and reconnect the tube by microsurgery.

Currently, tubal cannulation is typically performed by either hysteroscopy or fluoroscopy. With hysteroscopy, a telescope is introduced into the uterine cavity under local or general anesthesia. With fluoroscopy, a stent is guided into the tube under radiographic control. Of the two types of procedures, hysteroscopy is a more involved procedure both because the patients generally require some kind of sedation as well as the need to distend the uterus with gas or fluid in order to visualize the tubal ostium. This degree of involvement makes the procedure more expensive and subject to a greater potential for complications such as fluid overload, pulmonary edema, and potentially fatal embolism.

Physicians prefer the fluoroscopic procedure because the procedure is easier to perform and does not require anesthesia or uterine distension, therefore reducing the potential for complications as well having a lower cost. The cannulators presently available are rigid instruments with a predetermined curvature that is intended to accommodate the majority of tubal ostium locations within the uterus. The inventors, however, have determined that this “one size fits all” approach is unsuitable for all patients and a need exists for a more accommodating instrument.

Others have developed various instruments for accessing the uterine cavity for various medical purposes. U.S. Pat. No. 4,585,438 discloses a semi-rigid tubular member for injecting material, such as sperm, into the uterine cavity. The tubular member is characterized as being semi-rigid and flexible enough to bend with the natural curvature of the uterus while yet being rigid enough to maintain its shape.

U.S. Pat. No. 5,195,964 discloses a catheterization cannula for sealing the uterine cavity to allow injection of a radiopaque dye in the cavity. The catheter shaft portion is characterized as being flexible. The catheter shaft portion has an inner tuber 58 and an outer tube 60 described in reference to FIG. 3a as being made from a flexible material such as nylon. The purpose of the outer tube is to increase the rigidity of the cannula while still allowing some flexibility.

U.S. Pat. No. 6,007,478 is directed to tubes used with blood pumps, for example, during cardiovascular surgeries. The specification describes the tubes as having different degrees of flexibility along the length of the tube. This varying flexibility is achieved by using sections of tube joined in butt joints rather than step joints. The sections of tube are made using layers of plaster with different degrees of flexibility. By varying the thickness of the layer of one plastic versus another, the flexibility can be varied. The tubes may include spiral springs to prevent the collapse of the tube in use and radial deformation. The spring material may be subject to significant deformation but still regain its shape.

U.S. Pat. No. 6,491,645 is directed to a uterine tissue collector. The device is disclosed as being in the form of a cannula with a distal portion. The distal portion can be flexed or bent in different directions by using guide wires attached to a handle at a proximal end of the device. The cannula is characterized as preferably being formed from a flexible material and optionally including a portion with a low bending moment to allow for easy flexing of the distal portion. This may be accomplished by using notches in the wall of the cannula. The flexing or bending of the distal portion does not appear to be permanent but instead is subject to continued force applied by the guide wires.

US Patent Publication No. 2004/0054322 is directed to a shape-transferring cannula that includes two parallel rigidizing sections that alternatingly stiffen and relax with respect to one another and alternatingly transfer the path shape traced-out by the articulating tip to one another. A steerable articulated tip is attached to one of the rigidizing sections. The cannula's custom shape is formed by guiding the articulated tip along a desired path direction, stiffening the attached rigidizing section, and advancing the other rigidizing section along the stiffened section. The cannula, however, does not appear to be malleable and have its shape permanently altered.

### SUMMARY

In one general aspect, a cannulator has an adjustable shape for conforming to an anatomy to reach the tubal ostium of a patient. The cannulator includes a body segment, a tip section, and a handle. The body segment is made of a malleable material having a malleable characteristic with a degree of resistance to lateral deflection until sufficient force is applied to cause permanent bending thereby imparting a shape to the body segment. The tip section is positioned at a distal end of the body segment and the handle is positioned at a proximal end of the body segment.

Embodiments of the cannulator may include one or more of the following features. For example, the handle may be made of a plastic. The handle may include a visual reference to indicate an orientation of the tip section. The handle may include a circular shaft and the visual reference may include a first reference on the circular shaft and a second reference on the circular shaft, the positions of the first reference and the second reference defining the diameter of the circular shaft. The visual reference may include text that includes right and left directions. The handle may include two or more ports. The ports may be used for one or more of injecting a fluid, inserting a medical device, and withdrawing a fluid.

The body segment may include a wall defining a lumen within the body segment, the lumen passing between the handle and the tip section. The lumen may have an inner diameter of between 4 F and 6 F. The malleable material may be one or both of a malleable metal material and a malleable plastic material. The malleable metal material may be one or more of aluminum and stainless steel.

The tip section may have a smooth and rounded end. The tip section may include a cone that tapers from a proximal end to a distal end. The tip section may be a rounded plastic piece.

The body segment and/or the tip section may be coated with a coating.

In another general aspect there is provided a method of cannulating a fallopian tube. The method includes providing a tubal cannulator, adjusting a shape of the tubal cannulator and inserting the tubal cannulator into the uterine cavity and guiding the tip section to the tubal ostium. The tubal cannulator includes a body segment, a tip section, and a handle. The body segment is made of a malleable material having a malleable characteristic with a degree of resistance to lateral deflection until sufficient force is applied to cause permanent bending thereby imparting a shape to the body segment. The tip section is positioned at a distal end of the body segment and the handle is positioned at a proximal end of the body segment.

Embodiments of the method may include one or more of the following features or those described above. For example, the method may further include advancing a medical device through a lumen of the cannulator into the proximal tubal lumen. The method may further include removing the cannulator, modifying the shape of the cannulator, and reinserting the tubular cannulator. The malleable material may be one or both of a malleable metal material and a malleable plastic material. The malleable metal material may be one or more of aluminum and stainless steel.

In the method, adjusting a shape of the cannulator may include adjusting the shape of the body segment and/or the tip section.

The cannulator advantageously can be used under either laparoscopic or fluoroscopic control. It can eliminate the need for hysteroscopy to selectively cannulate each fallopian tube. This reduces operating time, the need for uterine distention, expensive monitoring/video equipment, and the possibility of major complications, such as fluid overload, pulmonary edema, and death. The cannulator also may permit most anastomoses to be performed laparoscopically rather than through a significantly more invasive laparotomy. In addition, while malleable, the cannulator nonetheless has enough strength to allow it to be advanced against the cervix adjacent to the tubal ostium, thus permitting easier visualization of that region during laparoscopy.

The details of various embodiments of the invention are set forth in the accompanying drawings and the description below. Other features and advantages of the invention will be apparent from the description, drawings, and claims.

### DESCRIPTION OF THE DRAWINGS

FIG. 1 is a plan view of the female reproductive system.

FIG. 2 is a close up plan view of a fallopian tube.

FIG. 3 is a perspective view of a first implementation of a malleable cannulator.

FIG. 4 is a perspective view of a second implementation of a malleable cannulator.

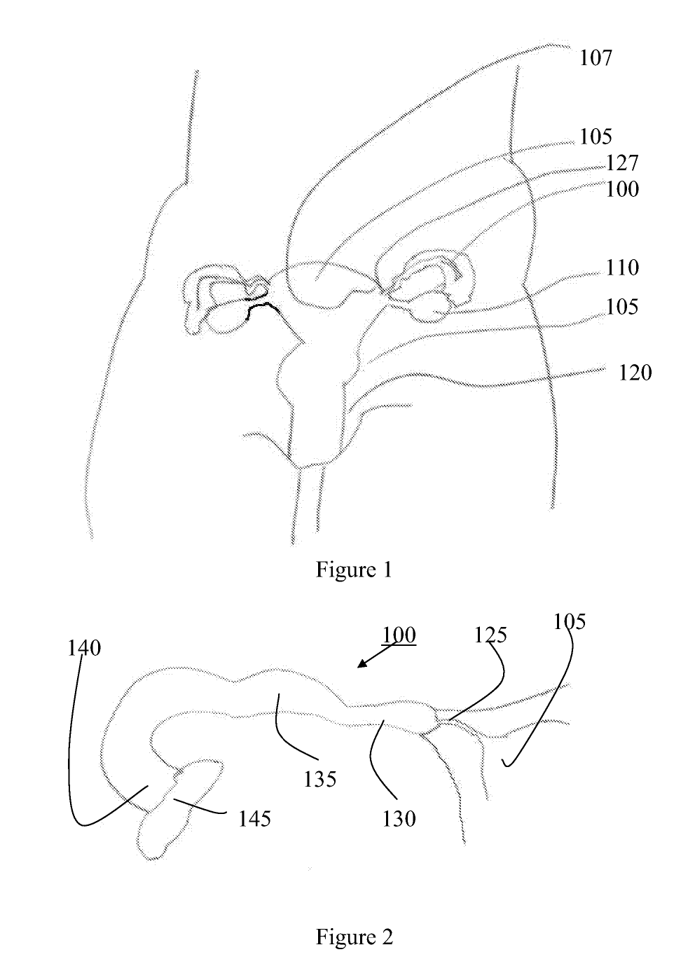

### DETAILED DESCRIPTION

The inventors have determined that the current cannulators used in fallopian tube cannulation do not permit universal fallopian tube cannulation because the cannulators are constructed from overly rigid plastics. To address this limitation, the inventors have developed a radiopaque malleable cannulator. The cannulator is constructed with a malleable material that allows the operator to adjust the cannulator's curvature in the procedure room to best fit the instrument to the patient's anatomy. Advantageously, the malleable material may be metal, plastic and or a composite of the two that is visible under x-ray (i.e., radiopaque) so that it can be utilized not only during surgery but also during fluoroscopic (i.e., x-ray) procedures as well. As used herein, the term malleable means the physical property of a material that allows the material to be deformed without cracking or at least without destructive cracking that causes the cannulator to be unusable for the intended purpose.

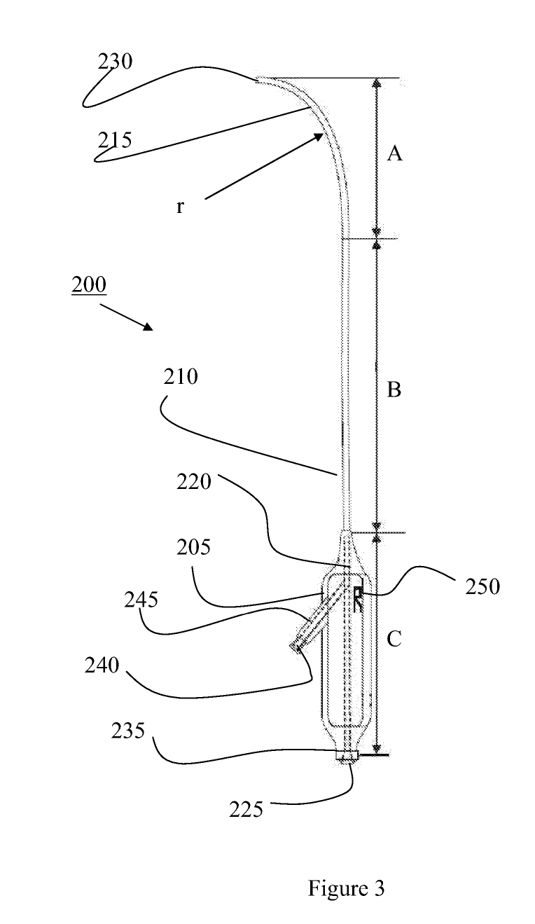

Referring to FIG. 3, a cannulator 200 includes a handle 205, a body segment 210, and a tip section 215. The body segment 210 is connected at a proximal end to the handle 205 and at a distal end to the tip section 215. The cannulator 200 has a lumen 220 that passes between a proximal opening 225 in the handle and a distal opening 230 in the tip section. The handle 205 includes a funnel luer lock 235 at the proximal end and a side injection luer lock 240 includes a lumen 245 that connects to the lumen 220. The handle 205 also includes an indicator 250. The indicator 250 can be, for example, a letter indicating the orientation of the handle 205 relative to the tip section 215. Specifically, the indicator on the handle can be for the orientation of the tip with respect to the patient. Thus, an indicator of “RIGHT” on the handle would be for a tip section oriented to the left with respect to the handle but oriented to the right with respect to the patient.

In some embodiments, the tip section 215 has a length, A, of between approximately 30 to 50 mm and, more particularly, approximately 40 mm. The tip section may have a radius, r, of between approximately 20 and 60 degrees, and more particularly, approximately 30 degrees. It should be noted that the radius of the tip section may be adjusted during use of the cannulator 200 to better fit an individual's anatomy. It is expect that most patients' anatomy will match a radius of approximately 30 degrees, the vast majority of patients' anatomy will match a radius of approximately 20 degrees to 60 degrees, and essentially all patients' anatomy will be matched with the ability to adjust the cannulator's curvature. It also should be noted that the tip section refers to the distal end of the cannulator and may not be a separate piece from the body segment. Thus, references to a tip section should be understood to have a meaning that includes a separate tip attached to the body segment, a region of the distal end of the cannulator and a section of the cannulator that extends from the body segment.

The body segment 210 has a length, B, of between approximately 120 mm and 160 mm and, more particularly, approximately 140 mm. The body segment 210 can have its shape adjusted as well. The handle 205 can have a length, C, of between approximately 50 mm and 70 mm and, more particularly, approximately 60 mm.

The lumen 220 has an inner diameter that is approximately 4-9 French (F) or approximately 1.5 mm to 3 mm inner diameter (where 1 F is equivalent to 0.33 mm). More particularly, the lumen 220 may have an approximately 5-6 F inner diameter. The outer diameter of the body segment 210 may be approximately three to five mm and the outer diameter of the tip 215 may be tapered and have an outer diameter of approximately three to five mm or smaller. The funnel luer lock 235 may have an inner diameter of approximately 2.5 mm and an outer diameter of approximately 3 mm. It should be noted that the dimensions for diameters of the luer lock are exemplary and other dimensions for the luer lock may be used.

The tip section 215 is configured to be atraumatic with a rounded, blunt or cone-shaped end that fits easily within the uterine corneal. The tip section can be formed by shaping the distal end of the body segment or by attaching a preformed tip to the body segment. For example, the tip section 215 can be made of an injection molded plastic that either is injection molded onto the cannulator, screwed onto the cannulator, or otherwise affixed to the cannulator. Alternatively, the tip section 215 can be integral with the cannulator and coated to make the tip section less traumatic.

The handle is configured to be easily gripped by the physician and optionally marked on opposite sides with an indicator 250 that may be a letter R or L, or the like. As noted above, the indicator 250 is used to indicate the orientation of the handle 205 relative to the tip section 215 with respect to the patient's anatomy. Such indicators are designed to be reference points to the physician using the cannulator. For example, if the physician has the tip section bent to the right while the handle side marked with an L is facing the physician, the physician can be aware during the procedure of the orientation of the tip relative to the handle by merely looking at the handle. Thus, if the cannulator is inserted into the patient and the handle side marked L is facing the physician, if the physician has followed the convention described above, the physician will know that the tip section is bent to the right relative to the handle and is therefore oriented to the patient's left.

The handle can be made of, for example, any biocompatible plastic. In particular, the plastic can be one that is easily injection molded, such as polyethylene and polypropylene, and injection molded onto the proximal end of body segment, screwed onto the proximal end of the body segment, or affixed to the proximal end of the body segment.

The body segment 210 and/or tip section 215 are made of any biocompatible, malleable metal, plastic or composite of the two. Examples of malleable metals include but are not limited to aluminum, stainless steel, silver, gold, silver-coated copper, and other alloys and composites. An example of a malleable composite is an extruded plastic tube in which part of the wall, either within the plastic or external to the plastic, includes a malleable metal portion. For example, a mesh of a malleable aluminum wire can be formed within the plastic wall using common extrusion techniques. The cannulator then can be bent and its bent shape retained during the procedure. The malleability of the body segment and/or tip section should be such that they can be bent by the physician yet not bend during insertion and advancement in the patient. The body segment 210 and tip section 215 may be made by one of several fabrication methods. For example, the body segment and tip section may be extruded separately and then attached or extruded as a single piece with the body segment integral with the tip section. To assemble the device, the body segment and/or tip section can be coated and then attached to a molded handle. Alternatively, the body segment and/or tip section may be coated after assembly. The tip section should be blunt and/or have extra coating or blunt tip attachment to be atraumatic.

The cannulator 200 may used by a physician in a variety of medical procedures, and, in particular, in the types of procedures described above, namely, tubal obstructions caused by mucous plugs and tubal obstructions classified as true obstructions. In either type of procedure, the cannulator first is shaped to provide a radius of curvature that the physician determines to be a suitable starting point. The cannulator may shaped at either or both of the tip section and the body segment. Then the cannulator is introduced into the uterine cavity through the cervix and the tip guided to the tubal ostium. If the curvature of the tip section is not correct, the physician removes the cannulator, modifies the shape of the body segment 210, reinserts the cannulator, and advances the cannulator to the tubal ostium.

Once the tip section of the cannulator is successfully advanced to the immediate proximity of the tubal ostium, a medical device, such as a stent or wire guide, is then pushed through the lumen of the cannulator and into the proximal tubal lumen. One example of the stent or wire guide that can be used is the Cook Medical Road Runner PC Wire Guide available from Cook Medical of Bloomington, Ind. The physician then can use the injection luer lock to inject a contrast agent or dye to verify patency or placement, or alternatively or in addition, inject a flushing solution to clear the lumen of debris, and withdraw a fluid or debris. It should be noted that the medical device used may be a stent or wire guide but may also be another type of medical device, such as a balloon, a laser fiber, a fiber optic device, or other device for visualizing the tubal site or opening the tubal site.

In cases of soft obstructions, referred to above as mucous or tubal plugs, the stent or wire guide dislodges the plug and opens the tube. In the true obstructions, also known as hard obstructions, the stent or wire guide stops at the level of the obstruction and the obstructed segment must be surgically removed, which leaves the tube separated into two segments apart from each other. The physician then advances the stent or wire guide from the first, or proximal, segment of the tube to the other, distal segment. The physician then can use the stent/wire guide as both a guide and a stable platform to visualize the tubal lumen and facilitate the placement of stitches to reconnect the tubal segments by forming an anastomosis of the two severed ends of the fallopian tube. The physician then can use the injection luer lock to flush the tubal segments and/or visualize the patency.

The cannulator can be used in other applications as well. For example, the cannulator can be used in selective chromotubation to check the tubal patency of each fallopian tube. The cannulator can be used to remove or flush amorphous material, such as tubal mucoid plugs, from the tubal lumen using the injection luer lock. This injection luer lock can be used to inject a contrast agent or dye to assist in visualizing the anatomy of the patient and the patency of the tubal lumen. The injection luer lock also can be used to inject fluids to flush the tubal lumen or assist in opening the lumen. Simultaneously, the funnel luer lock can be used to advance a medical device through the tubal cannulator. Advantageously, the cannulator 200 can be used in place of a number of devices and their various functions by opening the lumen, visualizing the lumen, etc., such that multiple devices do not need to be inserted and withdrawn.

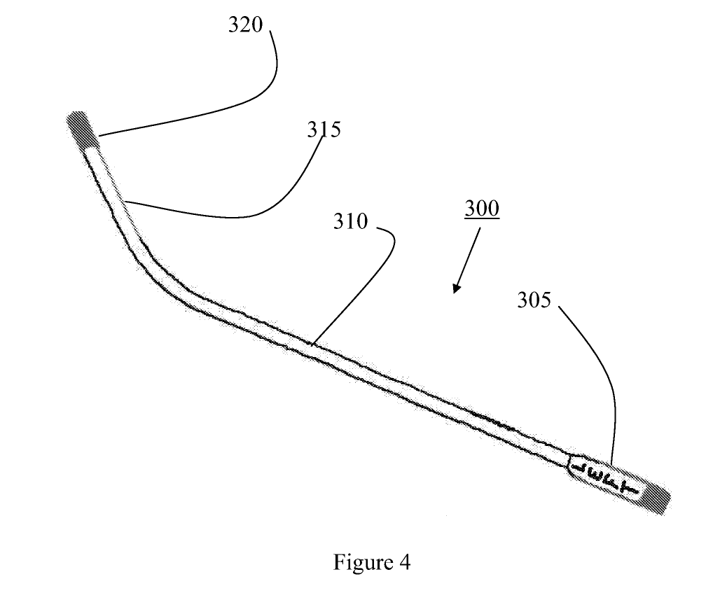

Referring to FIG. 4, in a second implementation of a cannulator, a cannulator 300 includes a handle 305, a body segment 310, a tip section 315 and a tip attachment 320. The handle, body segment and tip section are similar to the corresponding components in the cannulator 200. However, the cannulator 300 differs from the cannulator 200 by the inclusion of a tip attachment 320 attached to the tip section 315. The tip attachment 320 may be made of a biocompatible plastic that can be molded, screwed, affixed, or otherwise attached to the tip section 315 to cause the distal end of the cannulator 300 to be atraumatic. In this manner, the cannulator 300 does not need to be coated with an atraumatic coating. Nonetheless, one or more of the tip attachment 320, tip section 315 and body segment 310 may be coated to reduce any trauma with the tissue.

The cannulators 200 or 300 also can be used with an optional set of shaping mandrils. The shaping mandrils may be rigid metal rods that are configured to fit within the lumen 220 of the cannulator and impart a preset shape to the cannulator. For example, the shaping mandrils may be configured such that a set of, for example, up to three mandrils will provide the curves that will be needed in the vast majority of the procedures. The physician may initially insert the cannulator and determine that the initially selected shape forms too tight of a radius and that a looser radius is needed. By comparing the initially selected shape to the mandrils, the physician can easily make a minor adjustment to the curvature and reinsert the cannulator.

While several particular forms of the invention have been illustrated and described, it will be apparent that various modifications and combinations of the invention detailed in the text and drawings can be made without departing from the spirit and scope of the invention. For example, references to methods of construction, specific dimensions, shapes, utilities or applications are also not intended to be limiting in any manner and other materials and dimensions could be substituted and remain within the spirit and scope of the invention. Accordingly, it is not intended that the invention be limited, except as by the appended claims.

What is claimed is:

1. A cannulator having an adjustable shape for conforming to an anatomy to reach the tubal ostium of a patient, the cannulator comprising:
a body segment comprising a malleable material having a malleable characteristic with a degree of resistance to lateral deflection until sufficient force is applied to cause permanent bending thereby imparting a shape to the body segment;
a tip section at a distal end of the body segment; and
a handle at a proximal end of the body segment.

2. The cannulator of claim 1, wherein the handle includes a visual reference to indicate an orientation of the tip section.

3. The cannulator of claim 1, wherein the handle comprises a plastic.

4. The cannulator of claim 1, wherein the body segment comprises a wall defining a lumen within the body segment, the lumen passing between the handle and the tip section.

5. The cannulator of claim 4, wherein the lumen has an inner diameter of between 4 F and 6 F.

6. The cannulator of claim 1, wherein the malleable material comprises one or both of a malleable metal material or a malleable plastic material.

7. The cannulator of claim 6, wherein the malleable metal material comprises one or more of aluminum and stainless steel.

8. The cannulator of claim 1, wherein the tip section comprises a smooth and rounded end.

9. The cannulator of claim 1, wherein the tip section includes a cone that tapers from a proximal end to a distal end.

10. The cannulator of claim 1, wherein the tip section comprises a rounded plastic piece.

11. The cannulator of claim 1, wherein the body segment and/or the tip section are coated with a coating.

12. The cannulator of claim 2, wherein:
the handle comprises a circular shaft; and
the visual reference includes a first reference on the circular shaft and a second reference on the circular shaft, the positions of the first reference and the second reference defining the diameter of the circular shaft.

14. The cannulator of claim 1, wherein the visual reference comprises text that includes right and left directions.

15. The cannulator of claim 1, wherein the handle comprises two or more ports.

16. A method of cannulating a fallopian tube, the method comprising:
providing a tubal cannulator, the tubal cannulator including a body segment, a tip section and a handle, the body segment comprising a malleable material having a malleable characteristic with a degree of resistance to lateral deflection until sufficient force is applied to cause permanent bending thereby imparting a shape to the body segment, the tip section being positioned at a distal end of the body segment, and the handle being positioned at a proximal end of the body segment;
adjusting a shape of the tubal cannulator; and
inserting the tubal cannulator into the uterine cavity and guiding the tip section to the tubal ostium.

17. The method of claim 16, further comprising advancing a medical device through a lumen of the cannulator into the proximal tubal lumen.

18. The method of claim 16, further comprising removing the cannulator, modifying the shape of the cannulator, and reinserting the tubular cannulator.

19. The method of claim 16, wherein the malleable material comprises one or both of a malleable metal material or a malleable plastic material.

20. The method of claim 19, wherein the malleable metal material comprises one or more of aluminum and stainless steel.

21. The method of claim 16, wherein adjusting a shape of the cannulator comprises adjusting the shape of the body segment and/or the tip section.
